(19) United States
(12) Patent Application Publication
(10) Pub. No.: US 2004/0132690 A1
(43) Pub. Date: Jul. 8, 2004

(54) Compositions with controlled release of lactic acid at vaginal level

(76) Inventors: Carla Marcella Carmella, Pavia (IT); Maria Cristina Bonferoni, Cava Manara (Province of Pavia) (IT); Paolo Giunchedi, Cava Manara (Province of Pavia) (IT)

Correspondence Address: Caleb Pollack, Eitan Pearl Latzer & Cohen Zedek, Suite 1001, 10 Rockefeller Plaza, New York, NY, 10020, US

(21) Appl. No.: 10/476,642

(22) PCT Filed: Apr. 30, 2002
(86) PCT No.: PCT/EP02/04748

(30) Foreign Application Priority Data
May 4, 2001 (IT) MI01A000913

(51) Int. Cl. A61K 31/722

(52) U.S. Cl. 514/55

(57) ABSTRACT

Pharmaceutical composition for vaginal administration comprising a gel based on chitosan and lactid acid.

### FIELD OF THE INVENTION

The present invention refers to pharmaceutical compositions, for the treatment of bacterial vaginosis, comprising a gel based on chitosan and lactic acid.

### PRIOR ART

In the art, pathological situations that affect the vaginal mucosa are well known, such as bacterial infections, which give rise to a pathological increase of the pH and the destruction of the natural flora of lactobacilli which, in healthy vaginal mucosa, maintain an acid pH with the physiological production of lactic acid. An increase of the vaginal pH may also occur in post-menopause age (from 4-4.5 to as much as pH 7) with consequent possible colonising of the vaginal mucosa by pathogenic micro organisms and an Increased risk of local infections. Irritation of the mucosa, with consequent itching, tenderness and unpleasant smell are the main consequences of these pathological situations. The treatment of the vaginal mucosa with pharmaceutical products of a topical type is an indubitable advantage in comparison with treatment by means of systemic administration, since It allows the avoiding of the possible collateral effects typical of that type of administration. Among the possible topical compositions, creams and gels present notable advantages in comparison with other types of pharmaceutical products, such as good compliance in administration by the patient and, thanks to their Theological characteristics, ease of distribution of the pharmaceutical product on the surface of the vaginal mucosa. Gels in particular, on account of the high water content in their structure, present the further advantage of a hydrating and lubricating action, which is particularly useful in pathological situations characterised by dryness of the vaginal mucosa.

There was therefore a need for pharmaceutical compositions, in particular for topical application at vaginal level, for the treatment of various types of bacterial vaginosis and for the restoring (recolonising) of the physiological flora of lactobacilli having good biodegradability and biocompatibility with the vaginal environment, with a pH that falls within the range of physiological values of the vaginal cavity, having improved muco-adhesive properties and avoiding undesired contraceptive effects.

### SUMMARY OF THE INVENTION

A new pharmaceutical composition has now been discovered, which is biodegradable and biocompatible with the vaginal cavity, comprising a gel of chitosan and lactic acid for the treatment of various types of bacterial vaginosis and for the restoring (recolonising) of the physiological flora of lactobacilli; this composition releases lactic acid gradually (at a constant rate), over a prolonged period of time (controlled release), an acid which is physiologically present on the vaginal mucosa, with improved muco-adhesive properties and avoiding undesired contraceptive effects.

### BRIEF DESCRIPTION OF THE FIGURES

FIG. 1: curves of the release of lactic acid (mg/h) of the compositions according to the present invention, containing 1.65% by weight of lactic acid (examples 1 and 3) and of the composition for comparison (example 5) containing hydroxypropylmethylcellulose (HPMC) and 1.65% by weight of lactic acid.

FIG. 2: curves of the release of lactic acid (mg/h) of the compositions according to the present invention containing 3% by weight of lactic acid (examples 2 and 4) and of the composition for comparison (example 6) containing hydroxypropylmethylcellulose (HPMC) and 3% by weight of lactic acid.

FIG. 3: curves of variation of the pH value of the phosphate buffer solution used as a reference medium, following the release, in said solution, of lactic acid by the compositions described in the examples 1, 2, 3 and 4, and by the compositions for comparison described in the examples 5 and 6.

FIG. 4: pH values of the compositions described in the examples 1, 2, 3 and 4, and of the compositions for comparison described in the examples 5 and 6 before and after the test of the release of lactic acid.

FIG. 5: comparison among the curves of the release of lactic acid of the compositions described in the examples 1, 2, 3 and 4 and the curve of the release of lactic acid of the commercial composition LACTAL®.

FIG. 6: graphic representation of the muco-adhesive capacities, expressed in milliNewton (mN) as the detaching force of the compositions described in the examples 1, 2, 3 and 4 the commercial compositions for comparison LACTAL® and REPLENS® from vaginal mucosa obtained from a pig.

### DETAILED DESCRIPTION OF THE INVENTION

The object of the present invention is therefore a pharmaceutical composition for vaginal administration comprising a gel of chitosan and lactic acid.

In particular the weight ratio chitosan: lactic acid is between 1:1 and 2:1, and is preferably 1.8:1.

Said gel presents an improved muco-adhesive property, and the capacity of controlled release of lactic acid.

In particular the gel has a pH between 3 and 5, preferably between 3.5 and 4.5.

The gel, characterising the composition to which the present invention refers, has a chitosan concentration of between 1.5% and 5% of the weight, more preferably between 2% and 4% of the weight. The chitosan concentration of 3% by weightis the one most preferred.

The chitosan used in the preparation of the present compositions has a viscosity, measured for a solution of 1% by weight of chitosan in 1% acetic acid, comprised between 0.200 and 0.800 Pa.s at 37° C. and 20 s⁻¹. Preferably the viscosity of the chitosan is between 0.250 and 0.500 Pa.s at 37° C. and 20 s⁻¹; the viscosity of 0.300 Pa.s is particularly preferred.

In the present invention, the term “chitosan” is not limited to the product obtained by deacetylation of chitin, but encompasses any chitosan derivatives modified in order to improve their biocompatibility, biodegradability, or solubility; such derivatives are known in the art, and are e.g. partially hydrolysed chitosans, partially reacetylated chitosans, etc. Gels formed by mixing these derivatives with lactic acid are within the scope of the present invention.

The gel, which characterises the composition to which the present invention refers, has a concentration of lactic acid between 1% and 5% by weight, more preferably between 1.5% and 3% by weight. The concentration of lactic acid of 1.65% by weight is the one preferred.

The gel, which characterises the composition to which the present invention refers, has a viscosity between 2.00 Pa.s at 37° C. (20 s⁻¹) and 20.00 Pa.s a 37° C. (20 s⁻¹), preferably between 3.00 Pa.s at 37° C. (20 s⁻¹) and 15.00 Pa.s at 37° C. (20 s⁻¹). The gel, which characterises the composition to which the present invention refers, may comprise excipients with a stabilising action, preserving agents, diluting agents well known In the field of pharmaceutical compositions for topical application.

The pharmaceutical compositions according to the present invention comprise muco-adhesive gels formed by the combination, as basic components, of chitosan and lactic acid.

Chitosan (or poly 1-4 D-glucosamine) is a biodegradable and biocompatible polymer of natural origin, widely used in the pharmaceutical field as an excipient. It has basic characteristics, and is almost insoluble in water; due to its pH-dependent solubility it does not tend to gel in a neutral/alkaline watery environment.

Lactic acid (2-hydroxyproplonic acid) is a colourless or pale yellow syrupy liquid, which may be mixed with water as defined in the Official Italian Pharmacopeia (ed. X).

In the compositions to which the present invention refers, the active principle (lactic acid) is combined, in gel form, with chitosan, which gives the characteristics that make it bioadhesive and the capacity to control the release of lactic acid, influencing the rate and duration of release.

In particular it has been observed that the combination of chitosan and lactic acid in the presence of water determines the formation of the corresponding lactate, which profoundly modifies the characteristics of the original basic polymer (chitosane). Chitosan lactate is a polymer which gels In contact with water, determining the formation of a viscous gel. This gel assumes characteristics which make it particularly suitable for transmucosal administration, since it assumes the capacity to control the release of the lactic acid that it contains, for periods of time and at a rate which depend on the intrinsic characteristics of the composition such as the molecular-weight (or viscosity) of chitosan, the lactic acid: polymer weight ratio, the percentage weights of the two essential components of the gel; i.e. lactic acid and chitosan; it also acquires improved bioadhesive proprieties, which make it particularly suitable (considering the biocompatibility and biodegradability of chitosan) for remaining in contact with the surface of a mucosa for prolonged periods of time, such as those of prolonged release.

Pharmaceutical compositions comprising a gel with a base of chitosan and lactic acid, according to the present invention, are suitable for administration at vaginal level, in combination with lactic acid, also of other active principles, such as antimicrobic agents chosen from the group composed of antibacterial and/or antifungal agents.

The pharmaceutical compositions to which the present invention refers are obtained through processes of mixing and gelling that are well known In the field of production of drugs for topical application in the vaginal cavities.

The gel obtained in accordance with the present Invention can be administered as such or may be further formulated in other forms for vaginal administration, such as suppository or tablets. The techniques for preparing these delivery forms are known in the art. For example, in the case of suppositories, the gel of the invention can be dried by spray-drying, liophilisation, etc, admixed with suitable excipients such as semisynthetic triglycerids, and cast into the suitable shape.

In the case of suppository and tablets, the muco-adhesive properties develop when the composition is hydrated by the physiological vaginal fluids.

Below are given some examples of the present invention for illustrative purposes, without limitation.

### EXAMPLE 1

### Composition LL 165%

1.65 g of lactic acid and 3 g of chitosan with viscosity 0.302 Pa.s at 37° C. and 20 s⁻¹, measured for a solution with 1% by weight of chitosan In 1% acetic acid, are mixed together and brought to 100 g with water.

A gel is obtained having pH 4.3 and viscosity 4.11 Pa.s at 37° C. (20 s⁻¹).

### EXAMPLE 2

### Composition LL 3%

3 g of lactic acid and 3 g of chitosan with viscosity 0.302 Pa.s at 37° C. and 20 s⁻¹, measured for a solution with 1% by weight of chitosan in 1% acetic acid, are mixed together and brought to 100 g with water.

A gel is obtained having pH 3.5 and viscosity 4.53 Pa.s at 37° C. (20 s⁻¹).

### EXAMPLE 3

### Composition H 1.65%

1.65 g of lactic acid and 3 g of chitosan with viscosity 0.770 Pa.s at 37° C. and 20 s⁻¹ measured for a solution with 1% by weight of chitosan in 1% acetic acid, are mixed together and brought to 100 g with water.

A gel is obtained having pH 4.3 and viscosity 14.08 Pa.s at 37° C. (20 s⁻¹).

### EXAMPLE 4

### Composition H 3%

3 g of lactic acid and 3 g of chitosan with viscosity 0.770 Pa.s at 37° C. and 20 s⁻¹, measured for a solution with 1% by weight of chitosan in 1% acetic acid, are mixed together and brought to 100 g with water.

A gel is obtained having pH 3.5 and viscosity 12.17 Pa.s at 37° C. (20 s⁻¹).

### EXAMPLE 5

### For Comparison

### Composition HPMC 1.65%

3 g of hydroxypropylmethylcellulose (HPMC: Methocel K4M) and 1.65 g of lactic acid are mixed together and brought to 100 g with water.

A gel is obtained having pH 2.4 and viscosity 5.16 Pa.s at 37° C. at 20 s⁻¹.

### EXAMPLE 6

### For Comparison

### Composition HPMC 3%

3 g of hydroxypropylmethylcellulose (HPMC: Methocel K4M) and 3 g of lactic acid are mixed together and brought to 100 g with water.

A gel is obtained having pH 2.3 and viscosity 5.15 Pa.s at 37° C. at 20 s⁻¹.

### EXAMPLE 7

### Measuring the Rate of Release of Lactic Acid and the Variation of the pH Values

Similar quantities (about 8 g) of the compositions described in the examples from 1 to 6 were placed in cylindrical containers having a surface of 11.6 cm² and immersed in beakers containing 50 ml of phosphate buffer 0.05 M, pH 6.8. The beakers were incubated at 37° C. in a tipping bath at minimum speed. At defined intervals of time, 500 μl were taken for the dosing of lactic acid. At the same time the pH of the buffer was measured.

The concentration of lactic acid in the release medium was determined by means of HPLC.

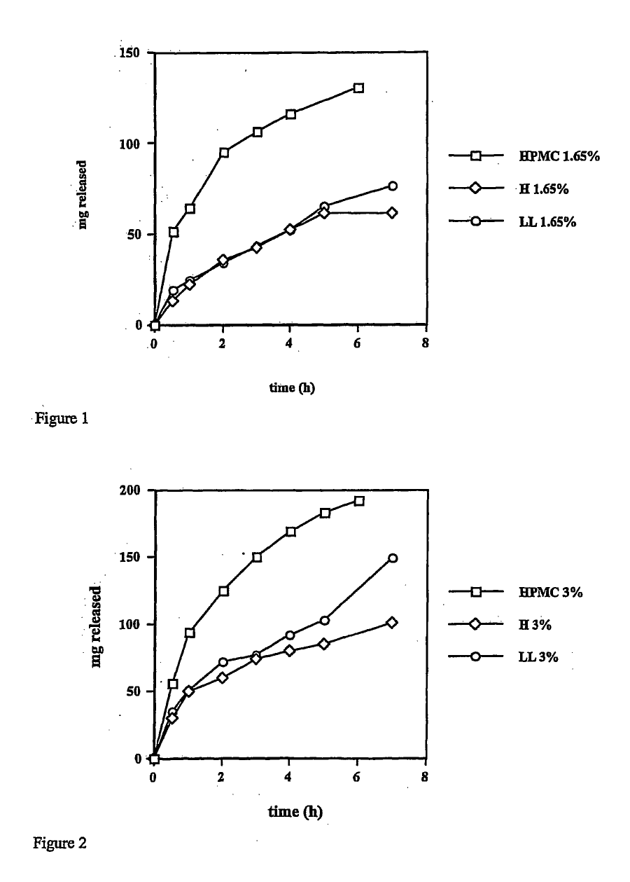

FIG. 1 shows the curves of the release of lactic acid of the compositions according to the present invention, containing 1.65% by weight of lactic acid (examples 1 and 3) and of the composition for comparison (example 5) containing hydroxypropylmethylcellulose (HPMC) and 1.65% by weight of lactic acid.

FIG. 2 shows the curves of the release of lactic acid (mg/h) of the compositions according to the present invention containing 3% by weight of lactic acid (examples 2 and 4) and of the composition for comparison (example 6) containing hydroxypropylmethylcellulose (HPMC) and 3% by weight of lactic acid.

The results show that the presence of chitosan allows a better control of the release of lactic acid with respect to the compositions for comparison containing hydroxypropylmethylcellulose.

The effect of the present compositions on the pH of the release environment is lo consistent with the profiles of release of lactic acid: the compositions with a base of HPMC lead to a more marked decrease of the pH with respect to those with a base of chitosan, which indicates a too sudden release. The compositions described In the examples from 1 to 4 with a base of chitosan, in which the release of lactic acid involves a smaller lowering of the pH of the buffer used as a release medium, are advantageous for avoiding undesired contraceptive effects (see FIG. 3).

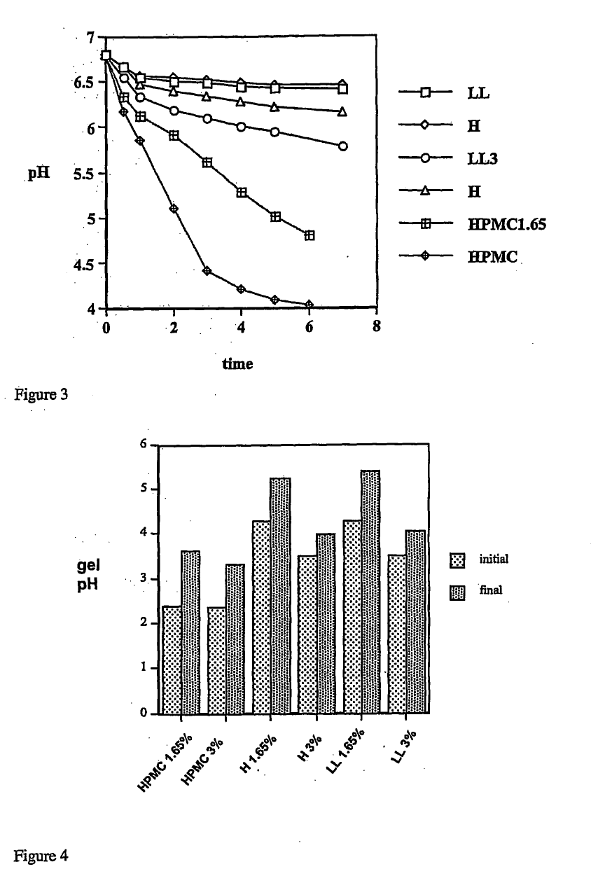

FIG. 4 shows the pH values, of the compositions described in the examples from 1 to 4 and of the compositions for comparison in examples 5 and 6 before and after the test of the release of lactic acid. The compositions in examples 1-4 with a base of chitosan all fall within the range of physiological values for the vaginal environment (3.5-5.0)

### EXAMPLE 8

### Comparison of the Profiles of the Release of Lactic Acid in Commercial Compositions

The comparison of the profiles of the release of lactic acid among the compositions described in the examples from 1 to 4 and a commercial composition INTILAC® or LACTAL® containing lactic acid, with a base of HPMC was carried out in Franz cells, using acetate buffer pH 5.0 as a release medium. At set intervals of time, amounts of 500 μl were, taken. The dosing of lactic acid was carried out with HPLC.

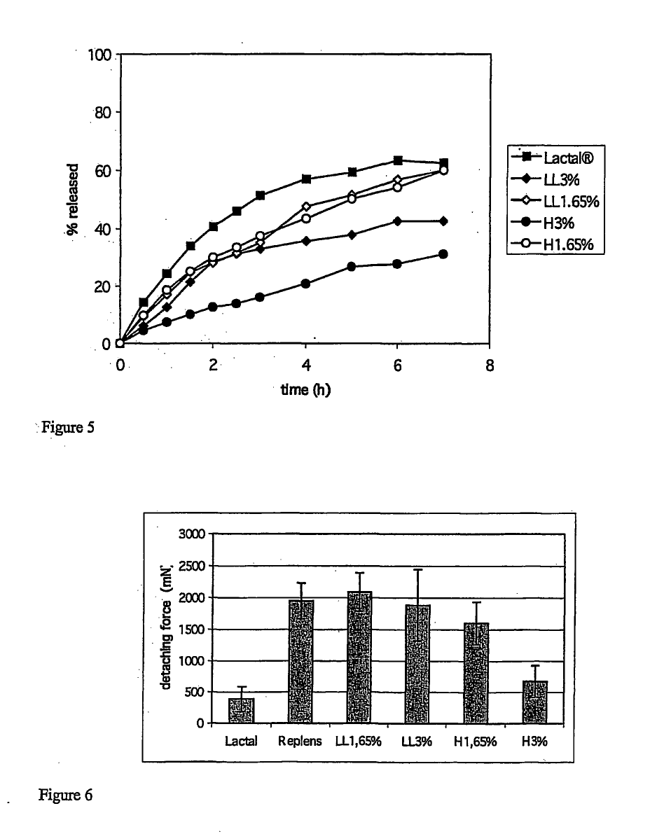

The results are shown in FIG. 5. The compositions described in the examples from 1 to 4 release the incorporated lactic acid in a way that is quantitatively comparable with the compared commercial composition. Said composition INTILAC® or LACTAL® marketed by Tremedic AB, Sweden, is a vaginal gel containing lactic acid but with a base of hydroxypropylcellulose and as excipients: water, propylene glycol, soda and glycogen. However, the compositions described in the examples from 1 to 4 present releases of lactic acid at more constant speeds, compatible with release prolonged over time.

### EXAMPLE 9

### Muco-Adhesive Capacities

The muco-adhesive capacity was assessed by measuring the detaching force between the compositions proposed and isolated pig vaginal mucosa.

The muco-adhesive effect is greater, the greater the detaching force. The detaching force is measured by placing In contact for 3 minutes a section of vaginal mucosa obtained from a pig and 100 mg of composition (gel) supported on a disc of filter paper which in turn is glued onto a cylindrical probe. After 3 minutes, the probe is moved away from the mucosa at a constant speed by an instrument which is able to measure the force necessary (in mN) to detach the gel from the mucosa.

For comparison the commercial compositions INTILAC® or LACTAL® were assessed in the same way, and also a muco-adhesive gel (not containing lactic acid, based on polyacrylic acid) intended for the hydration of the vaginal environment, REPLENS®.

The results are shown in FIG. 6. In all cases the compositions proposed with a base of chitosan and lactic acid show muco-adhesive capacities greater than those of INTILAC® or LACTAL®, and some of them (LL1.65% and LL 3%) are comparable with those of REPLENS®. It may be noted that the muco-adhesive capacity decreases as the molecular weight of the chitosan increases.

1. Pharmaceutical composition for vaginal administration comprising a gel based on chitosan and lactic acid.

2. Pharmaceutical composition according to claim 1 in which the weight ratio chitosan: lactic acid is between 1:1 and 2:1.

3. Pharmaceutical composition according to claim 2 in which the weight ratio is 1.8:1.

4. Pharmaceutical composition according to claims 1-3 in which the gel has a pH between 3 and 5.

5. Pharmaceutical composition according to claim 4 In which the pH is between 3.5 and 4.5.

6. Pharmaceutical composition according to claims 1-5 in which the gel has a concentration of lactic acid between 1% and 5% by weight.

7. Pharmaceutical composition according to claim 6 in which the gel has a concentration of lactic acid between 1.5% and 3% by weight.

8. Pharmaceutical composition according to claim 7 in which the gel has a concentration of lactic acid of 1.65% by weight.

9. Composition according to claims 1-8 in which the chitosan has a viscosity, measured for a solution of 1% by weight of chitosan in 1% acetic acid, comprised between 0.200 and 0.800 Pa.s at 37° C. and 20 s⁻¹.

10. Pharmaceutical composition according to claim 9 in which the viscosity of the chitosan, is between 0.250 and 0.500 Pa.s at 37° C. and 20 s⁻¹.

11. Pharmaceutical composition according to claim 10 in which the viscosity of the chitosan measured for a solution of 1% by weight of chitosan in 1% acetic acid is 0.300 Pa.s at 37° C. and 20 s⁻¹.

12. Composition according to claims 1-11 in which the gel has a concentration of chitosan between 1.5% and 5% by weight.

13. Pharmaceutical composition according to claim 12 in which the gel has a concentration of chitosan between 2% and 4% by weight.

14. Pharmaceutical composition according to claim 13 in which the gel has a concentration of chitosan of 3% by weight.

15. Pharmaceutical composition according to claims 1-14 in which the gel has a viscosity between 2.00 Pa.s at 37° C. (20 s⁻¹) and 20.00 Pa.s at 37° C. (20 s⁻¹).

16. Pharmaceutical composition according to claim 15 in which the gel viscosity is between 3.00 Pa.s at 37° C. (20 s⁻¹) and 15.00 Pa.s at 37° C. (20 s⁻¹).

17. Pharmaceutical composition according to claims 1-16 In which the gel is combined with suitable excipients and/or pharmaceutically compatible diluting agents.

18. Pharmaceutical composition according to claims 1-17, wherein the gel is dried and further processed into a vaginal suppository or a vaginal tablet.

19. Pharmaceutical composition according to claims 1-18 for the prevention and treatment of vaginal Infections.

20. Pharmaceutical composition according to claim 19 for the treatment of bacterial vaginosis and for the restoring (recolonising) of the physiological flora of lactobacilli.

21. Pharmaceutical composition according to claims 1-20 combined with antimicrobic agents chosen from the group composed of antibacterial and/or antifungal agents.

22. Use of a gel based on chitosan and lactic acid in the preparation of a pharmaceutical composition for the prevention and treatment of vaginal infections.

23. Use according to claim 22 for the treatment of bacterial vaginosis and for the restoring (recolonising) of the physiological flora of lactobacilli.
